(19) United States
(12) Patent Application Publication
(10) Pub. No.: US 2020/0321086 A1
(43) Pub. Date: Oct. 8, 2020

(54) DATA AGGREGATION IN HEALTH CARE SYSTEMS

(71) Applicants: Infinite Computer Solutions Inc., Rockville, MD (US); INFINITE COMPUTER SOLUTIONS (INDIA) LIMITED, Bangalore (IN)

(72) Inventors: Sanjesh Rao, Haymarket, VA (US); Harish PAI, Ashburn, VA (US); Mohamed Madar MOHIDEEN M Z, Chennai (IN)

(21) Appl. No.: 16/753,092

(22) PCT Filed: Oct. 3, 2018
(86) PCT No.: PCT/US2018/054125

§ 371 Date: Apr. 2, 2020

Related U.S. Application Data
(60) Provisional application No. 62/567,342, filed on Oct. 3, 2017.

(51) Int. Cl. G16H 10/60; G16H 40/20; G16H 80/00; G06F 21/62; G06F 16/2455

(52) U.S. Cl. CPC G16H 10/60; G16H 40/20; G06F 16/24556; G06F 21/6245; G16H 80/00

(57) ABSTRACT

According to an aspect of the present disclosure, a healthcare system maintains mapping data specifying the Electronic Medical Record (EMR) systems at which EMRs linked to healthcare providers are stored, each EMR containing information related to a corresponding patient. Upon receiving a request from a healthcare provider to view information related to a patient, the mapping data is examined to identify a set of EMR systems that store a set of EMRs that are linked to the healthcare provider and contain information related to the patient. The healthcare system then provides access to the identified set of EMRs using a common user interface. Accordingly, the healthcare system facilitates data aggregation from multiple EMR systems.

### CROSS-REFERENCE TO RELATED APPLICATIONS

This application claims the benefit of priority of U.S. Provisional Application No. 62/567,342, filed Oct. 3, 2017, the content of which is incorporated herein by reference in its entirety.

### TECHNICAL FIELD

Embodiments of the present disclosure relate generally to healthcare systems and in particular to data aggregation in such systems.

### BACKGROUND

Healthcare systems aid the delivery of various healthcare aspects such as work-flow coordination, records managements, diagnosis, etc., as is well known in the relevant arts. Healthcare systems are often employed by hospitals (healthcare delivery parties, in general) to aid various parties such as the medical staff and patients.

Data aggregation refers to the process of gathering information related to an entity of interest from different data sources and providing the gathered information together to users. Additional steps may be performed as part of the aggregation such as converting to a common format, generating a summary of the gathered information for ease of analysis, etc.

In healthcare systems, the entity of interest is typically a patient and as such the gathered information is in the form of electronic medical record (EMR) or electronic health records (EHR). In the following description, the term EMR is used to refer to both electronic medical records and electronic health records.

Aspects of the present disclosure are directed to EMR aggregation in healthcare systems, in particular when the EMR data is maintained in multiple EMR systems.

### BRIEF SUMMARY

According to an aspect of the present disclosure, a healthcare system maintains mapping data specifying the Electronic Medical Record (EMR) systems at which EMRs linked to healthcare providers are stored, each EMR containing information related to a corresponding patient. Upon receiving a request from a healthcare provider to view information related to a patient, the mapping data is examined to identify a set of EMR systems that store a set of EMRs that are linked to the healthcare provider and contain information related to the patient. The healthcare system then provides access to the identified set of EMRs using a common user interface. Accordingly, the healthcare system facilitates data aggregation from multiple EMR systems.

According to another aspect of the present disclosure, a healthcare system maintains a visit summary data specifying the details of interactions between patients and healthcare providers. Upon identifying based on the visit summary data, a set of patients having interactions with the healthcare provider, the healthcare system provides (e.g. displays on a display unit) the identified set of patients to the healthcare provider. The healthcare provider is accordingly facilitated to request and view information related to any patient of interest among the set of patients.

According to one more aspect of the present disclosure, when the information for different patients are stored on different EMR systems, a healthcare system displays different icons indicating the corresponding different EMR systems where the information is stored. In one embodiment, the healthcare system displays a first icon representing a first EMR system in association with a first patient in view of the information of the first patient being stored in the first EMR system, and displays a second icon representing a second EMR system in association with a second patient in view of the information of the second patient being stored in the second EMR system.

According to yet another aspect of the present disclosure, a healthcare system displays both of a first portion and a second portion of the information for a patient together as part of the common user interface even when the first portion and the second portion are stored respectively on different EMR systems. As such, the users/healthcare providers are provided with drill-down capabilities wherein the user is enabled to view the aggregated EMR data/patient information at various levels (e.g. hospital/transaction level) though the data at such levels may be maintained in different EMR systems.

According to another aspect of the present disclosure, a healthcare system facilitates a healthcare provider to collaborate with different sets of healthcare providers having responsibility for the patient, though the different sets are indicated by different portions of the patient information stored in different EMR systems.

According to an aspect of the present disclosure, a healthcare provider using a client system sends a request to view information related to a patient and in response provided access to a set of Electronic Medical Records (EMRs) containing information related to the patient using a common user interface. The healthcare provider is accordingly facilitated to access data stored in different EMR systems via a common user interface.

According to one more aspect of the present disclosure, a healthcare provider is first provided at a client system, a set of patients having interactions with the healthcare provider. The healthcare provider is accordingly facilitated to request and view information related to any patient of interest among the set of patients.

According to yet another aspect of the present disclosure, a healthcare provider using a client system is shown different icons for different patients, the different icons indicating the corresponding different EMR systems where the information of the patients is stored.

According to another aspect of the present disclosure, a healthcare provider using a client system is shown both of a first portion and a second portion of the information for a patient together as part of the common user interface even when the first portion and the second portion are stored respectively on different EMR systems.

According to another aspect of the present disclosure, a healthcare provider using a client system is facilitated to collaborate with different sets of healthcare providers having responsibility for the patient, though the different sets are indicated by different portions of the patient information stored in different EMR systems.

According to an aspect of the present disclosure, a third-party digital processing system (external to the healthcare system) maintains mapping data specifying the Electronic Medical Record (EMR) systems at which EMRs linked to healthcare providers are stored, each EMR containing information related to a corresponding patient. Upon receiving a request from a healthcare provider to view information related to a patient, the mapping data is examined to identify a set of EMR systems that store a set of EMRs that are linked to the healthcare provider and contain information related to the patient. The digital processing system then provides access to the identified set of EMRs using a common user interface. Accordingly, the third-party (external) digital processing system is configured to facilitate data aggregation from multiple different EMR systems.

According to another aspect of the present disclosure, a third-party digital processing system (external to the healthcare system) maintains a visit summary data specifying the details of interactions between patients and healthcare providers. Upon identifying based on the visit summary data, a set of patients having interactions with the healthcare provider, the digital processing system provides (e.g. displays on a display unit) the identified set of patients to the healthcare provider. The healthcare provider is accordingly facilitated to request and view information related to any patient of interest among the set of patients.

According to yet another aspect of the present disclosure, a third-party digital processing system (external to the healthcare system) displays different icons indicating the corresponding different EMR systems where the patient related information is stored.

According to one more aspect of the present disclosure, a third-party digital processing system (external to the healthcare system) displays both of a first portion and a second portion of the information for a patient together as part of the common user interface even when the first portion and the second portion are stored respectively on different EMR systems.

According to an aspect of the present disclosure, a third-party digital processing system (external to the healthcare system) facilitates a healthcare provider to collaborate with different sets of healthcare providers having responsibility for the patient, though the different sets are indicated by different portions of the patient information stored in different EMR systems.

Several aspects of the invention are described below with reference to examples for illustration. However, one skilled in the relevant art will recognize that the disclosure can be practiced without one or more of the specific details or with other methods, components, materials and so forth. In other instances, well-known structures, materials, or operations are not shown in detail to avoid obscuring the features of the disclosure. Furthermore, the features/aspects described can be practiced in various combinations, though only some of the combinations are described herein for conciseness.

While the features of the present disclosure are described substantially in the context of healthcare services, it should be appreciated that several aspects of the present disclosure can be applied to other industries such as banking, telecommunication, transport, education and retail services.

### BRIEF DESCRIPTION OF THE DRAWINGS

Example embodiments of the present disclosure will be described with reference to the accompanying drawings briefly described below.

FIGS. 1A-1C depicts example environments in which several aspects of data aggregation in healthcare systems can be implemented.

FIG. 2 is a block diagram of an example computing system in which several aspects of data aggregation in healthcare systems can be implemented.

FIG. 3 is a flowchart illustrating the manner in which data aggregation is provided in a healthcare system according to an aspect of the present disclosure.

FIGS. 4 and 5 depicts portions of a common graphical user interfaces (GUI) providing data aggregation from multiple EMR systems in an embodiment.

FIG. 6A depicts portions of a visit summary data specifying the details of interactions between patients and healthcare providers in one embodiment.

FIG. 6B depicts portions of a mapping data specifying the EMR systems linked to healthcare providers in one embodiment.

FIG. 7 is a block diagram illustrating an example implementation of a healthcare system.

FIG. 8 is a block diagram illustrating the manner in which a healthcare system is provided using a cloud infrastructure in an embodiment.

FIG. 9 is a block diagram illustrating the details of a digital processing system in which various aspects of the present disclosure are operative by execution of appropriate executable modules.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE EMBODIMENTS OF THE DISCLOSURE

### 1. Example Environments

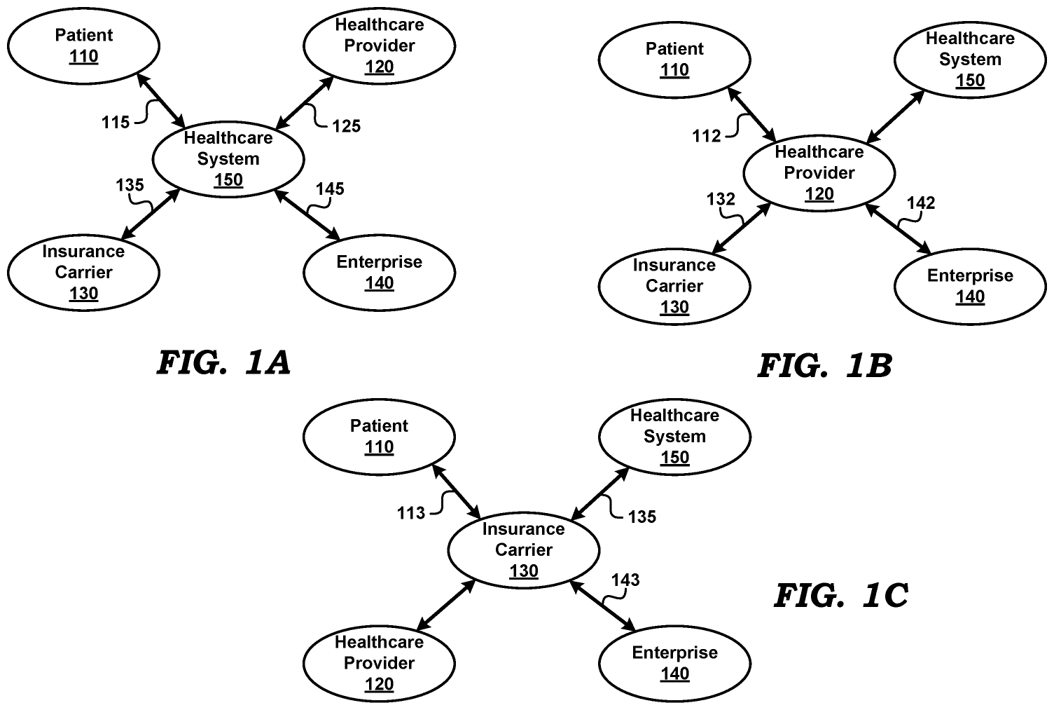

FIGS. 1A-1C depicts example environments in which several aspects of data aggregation in healthcare systems can be implemented. The example environments are shown containing entities/elements such as patient 110, healthcare provider 120, insurance carrier 130, enterprise 140 and healthcare system 150. The elements are shown interconnected by data links 115, 125, 135, 145, etc.

Merely for illustration, only representative number/type of elements/links is shown in the Figure. Many environments often contain many more elements and/or links, both in number and type, depending on the purpose for which the environment is designed. Each element and data link of FIGS. 1A-1C is described below in further detail.

Patient 110 refers to a recipient of healthcare services. Information on patient 110 is commonly maintained in the form of electronic medical/health records (EMR/EHRs), as is well known in the relevant arts. EMR/EHRs (hereinafter referred commonly as EMRs) may specify details such as patient identity, dates of visit to avail healthcare services, symptoms of health problems faced by the patient, lab reports, etc.

Healthcare provider 120 refers to a physician/doctor, nurse, medical staff, care giver, etc. who provide one or more healthcare services to the patient. Healthcare providers are commonly associated with a healthcare organization (hospital, clinic, etc.) located at one or more geographical locations. Each healthcare provider commonly has responsibility for one or more patients.

Insurance carrier 130 represents an organization that offers various health insurance plans to patients, with each insurance plan being designed to protect the patient financially when health problems arise. In the below description, it is assumed that patient 110 has subscribed to at least one insurance plan offered by insurance carrier 130.

Enterprise 140 represents a third party organization that is related to providing healthcare or management services (e.g. customer relationship management). Enterprise 140 generally operates data and applications independent of the other elements noted here.

Healthcare system 150 represents a system that assists a healthcare organization in the delivery of healthcare services to patients. Healthcare system 150 aids in various healthcare aspects such as work-flow coordination between healthcare providers and patients, records (such as EMR) management, diagnosis, etc., as is well known in the relevant arts.

Referring to FIG. 1A, healthcare system 150 maintains mapping data specifying the Electronic Medical Record (EMR) systems at which EMRs linked to healthcare providers are stored, each EMR containing information related to a corresponding patient. Healthcare system 150 then receives a request from healthcare provider 120 to view information related to patient 110.

In one embodiment, the request is received from healthcare provider 120 (via data link 125) such as a physician/doctor. The request may be received when the physician wants to review the status of his/her patients or in response to patient 110 coming for check-up/review with the physician, either in-house or as an out-patient at a premise of the healthcare organization as is well known.

In alternative embodiments, the request may be received from patient 110 via data link 115, for example, when the patient wishes to discuss health related issues such as symptoms, diagnosis, medicines, etc. with any healthcare providers of the healthcare organization. The request may also be received from insurance carrier 130 via data link 135 or from enterprise 140 via data link 145, for example, when the insurance carrier/enterprise wishes to review the condition of a specific patient with the healthcare providers related to the patient.

Healthcare system 150 examines the mapping data to identify a set of EMR systems that store a set of EMRs that are linked to the healthcare provider and contain information related to the patient. Healthcare system 150 then provides access to the identified set of EMRs using a common user interface (via data link 125). Thus, healthcare provider 120 is facilitated to access EMR data stored in multiple different EMR systems.

According to an aspect of the present disclosure, healthcare system 150 also maintains a visit summary data specifying the details of interactions between patients and healthcare providers. Upon identifying based on the visit summary data, a set of patients having interactions with healthcare provider 120, healthcare system 150 provides (e.g. displays on a display unit) the identified set of patients to healthcare provider 120. Healthcare provider 120 is accordingly facilitated to request and view information related to any patient of interest (such as patient 110) among the set of patients.

According to another aspect of the present disclosure, healthcare system 150 facilitates healthcare provider 120 to collaborate with different sets of healthcare providers having responsibility for the patient, though the different sets are indicated by different portions of the patient information stored in different EMR systems.

Referring to FIG. 1B, healthcare provider 120 sends a request (for example, using a client system) to view information related to patient 110 and in response provided access to a set of Electronic Medical Records (EMRs) containing information related to the patient using a common user interface. Healthcare provider 120 is accordingly facilitated to access data stored in different EMR systems via a common user interface. The request may be sent in response to receiving (via data link 112) a request from patient 110 for check-up/review with the healthcare provider. In alternative embodiments, the request may be sent in response to receiving corresponding requests for reviewing the condition of patient 110 from insurance carrier 130 (via data link 132) or from enterprise 140 (via data link 142).

Referring to FIG. 1C, insurance carrier 130 (a third-party different from the first party healthcare system 150 and the second party healthcare provider 120) maintains mapping data specifying the Electronic Medical Record (EMR) systems at which EMRs linked to healthcare providers are stored, each EMR containing information related to a corresponding patient. The EMR data may be stored in healthcare system 150 (via data link 135) and/or enterprise 140 (via data link 143). Upon receiving a request from patient 110 (via data link 113), insurance carrier 130 identifies a set of EMR systems that store a set of EMRs that are linked to healthcare provider 120 and contain information related to patient 110. Insurance carrier 130 then provides (e.g. displayed on a display screen) access to the identified set of EMRs using a common user interface.

Thus, the various entities/elements shown in FIGS. 1A-1C such as patient 110, healthcare provider 120, insurance carrier 130, enterprise 140 and healthcare system 150 operate to provide data aggregation in a healthcare system. The manner in which several features of the present disclosure can be implemented using a computing system (system architecture) is described below with examples.

### 2. System Architecture

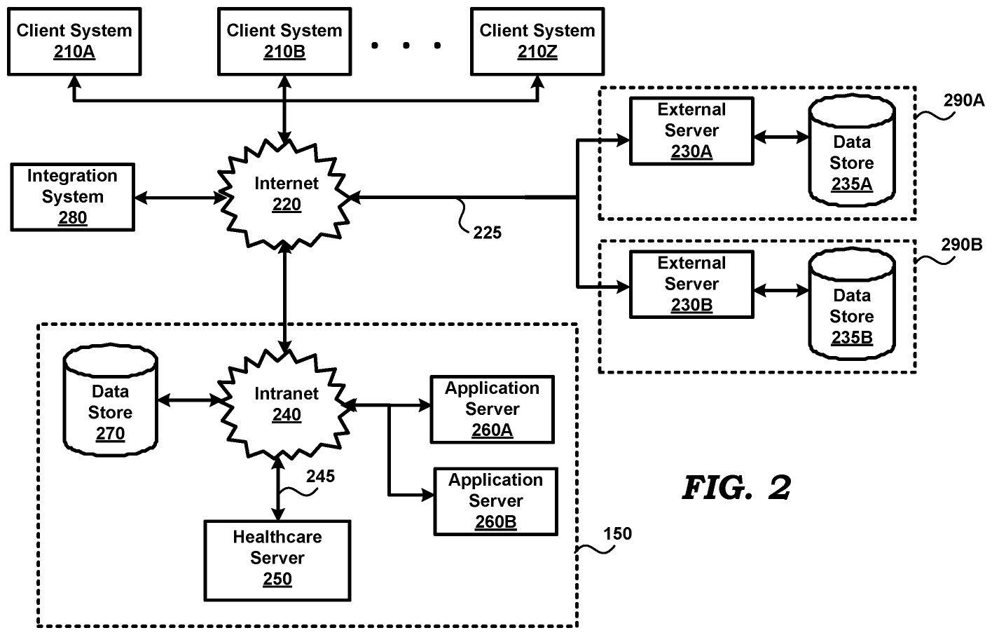

FIG. 2 is a block diagram of an example computing system in which several aspects of data aggregation in healthcare systems can be implemented. The block diagram is shown containing client systems 210A-210Z, Internet 220, Intranet 240, external servers 230A-230B, data stores 235A-235B & 270, application servers 260A-260B, healthcare server 250 and integration system 280.

In one embodiment, healthcare server 250, application servers 260A-260B, and data store 270 along with intranet 240 operate together to as healthcare system 150 (as indicated by the dashed rectangle) that provides effective collaboration among various healthcare providers such as physicians/doctors, medical staff/nurse, care providers, etc.

Each of systems 290A (containing external server 230A and data store 235A) and 290B (containing external server 230B and data store 235B) represents a corresponding third-party external system with which healthcare system 150 interacts with to provide various aspects of the present disclosure. For example, external system 290A may represent a system provided by insurance carrier 130, with external server 230A being an insurance server belonging to insurance carrier 130. The insurance server may maintain (in data store 235A) information on patients having insurance coverage with insurance carrier 130. External system 290B may represent a system provided by enterprise 140, with external server 230B being an enterprise server belonging to enterprise 140.

Merely for illustration, only representative number/type of systems/devices are shown in the Figure. Many environments often contain many more systems and/or devices, both in number and type, depending on the purpose for which the environment is designed. Each system/device of FIG. 2 is described below in further detail.

Each of client systems 210A-210Z represents a system such as a personal computer, workstation, mobile station, mobile phones, computing tablets, etc., used by users/healthcare providers to send user requests to applications executing in healthcare system 150 (such as in application servers 260A-260B or healthcare server 250), and display the corresponding responses. The responses may be in the form of web pages and thus the requests may be in the form of Uniform Resource Locators (URLs) with appropriate additional content to specify the user requests. The users may generate the user requests based on appropriate user interfaces provided on each client system. In one embodiment, client systems 210A-210Z runs on different operating systems such as ANDROID™ and IOS™, and provide user interfaces using different web browser applications such as INTERNET EXPLORER®, SAFARI™ FIREFOX® and CHROME®.

Intranet 240 represents a network providing connectivity between application servers 260A-260B, healthcare server 250 and data store 270, all provided as part of healthcare system 150. Internet 220 extends the connectivity of these (and other systems of the healthcare system) with external systems such as client systems 210A-210Z and external systems 290A-290B. Each of intranet 240 and Internet 220 may be implemented using protocols such as Transmission Control Protocol (TCP) and/or Internet Protocol (IP), well known in the relevant arts. In general, in TCP/IP environments, an IP packet is used as a basic unit of transport, with the source address being set to the IP address assigned to the source system from which the packet originates and the destination address set to the IP address of the destination system to which the packet is to be eventually delivered.

A (IP) packet is said to be directed to a destination system when the destination IP address of the packet is set to the (IP) address of the destination system, such that the packet is eventually delivered to the destination system by Internet 220 and intranet 240. When the packet contains content such as port numbers, which specifies the destination application, the packet may be said to be directed to such application as well. The destination system may be required to keep the corresponding port numbers available/open, and process the packets with the corresponding destination ports. Each of Internet 220 and intranet 240 may be implemented using any combination of wire-based or wireless mediums.

Each of data stores 235A-235B and 270 represents a non-volatile (persistent) storage facilitating storage and retrieval of a collection of data by applications executing in corresponding server systems. For example, data store 270 facilitates applications executing in application servers 260A-260B and healthcare server 250 to store/retrieve data related to patients, healthcare providers, the healthcare system, etc.

In one embodiment, each of data stores 235A-235B and 270 maintains information on patients in the form of EMRs. As noted above, EMRs may specify details of patients including patient identity, dates of visit, symptoms of medical problem faced by the patient, lab reports, patient records, etc. Each data store may also store the conversations of the participants of chat/interactive sessions between the various healthcare providers, the clinical data of the patients, etc.

Some of data stores 235A-235B and 270 may be implemented as a database server using relational database technologies and accordingly provide storage and retrieval of data using structured queries such as SQL (Structured Query Language). Some of data stores 235A-235B and 270 may be implemented as a file server providing storage and retrieval of data in the form of files organized as one or more directories, as is well known in the relevant arts.

Each of application servers 260A-260B, external servers 230A-230B and healthcare server 250 represents a server system, such as a web/application server, executing applications performing tasks requested by users (for example, using one of client systems 210A-210Z). Each server system may use data stored internally (for example, in a non-volatile storage/hard disk within the server system), external data (e.g., maintained in data store 235A-235B/270) and/or data received from external sources (e.g., from the user) in performing the requested tasks. The server system then sends the result of performance of the tasks to the requesting client system (e.g., one of 210A-210Z). The results may be accompanied by specific user interfaces (e.g., web pages) for displaying the results to the requesting user.

Integration system 280 represents a server system that that facilitates healthcare server 250 to interact with external systems 290A-290B. Integration system 280 may accordingly provide integration APIs (Application Programming Interface) containing a library of interfaces/protocols to external systems, an integration database for storing information sent/received from external systems, and a web server that facilitates interaction with external systems that operate with text based protocols such as Hyper Text Transfer Protocol Secure (HTTPS).

Healthcare server 250, provided according to several aspects of the present disclosure, facilitates aggregation of EMR data stored in multiple EMR systems (data stores noted above). The manner in which healthcare server 250 (and accordingly healthcare system 150) facilitates data aggregation is described below with examples.

### 3. General Flow

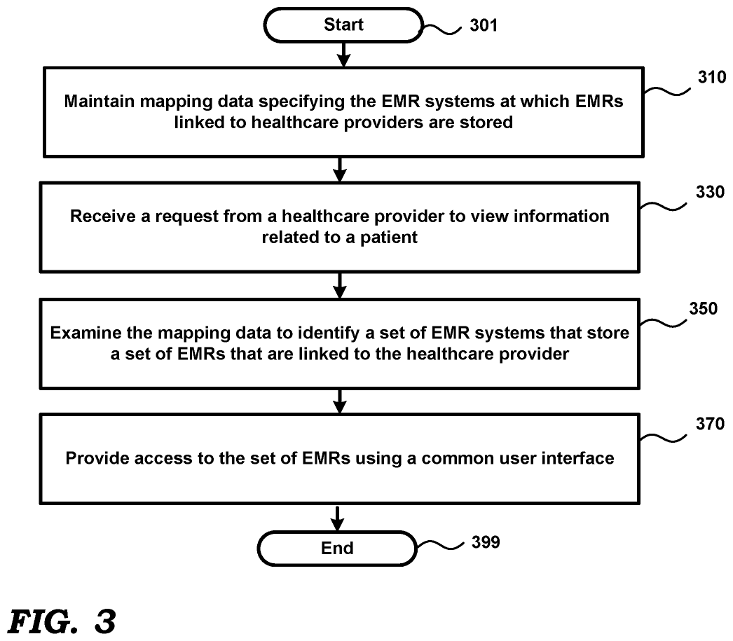

FIG. 3 is a flowchart illustrating the manner in which data aggregation is provided in a healthcare system according to an aspect of the present disclosure. The flowchart is described with respect to healthcare server 250 of FIG. 2 merely for illustration. However, the features can be implemented in other systems and environments also without departing from the scope and spirit of various aspects of the present disclosure, as will be apparent to one skilled in the relevant arts by reading the disclosure provided herein.

In addition, some of the steps may be performed in a different sequence than that depicted below, as suited to the specific environment, as will be apparent to one skilled in the relevant arts. Many of such implementations are contemplated to be covered by several aspects of the present disclosure. The flow chart begins in step 301, in which control immediately passes to step 310.

In step 310, healthcare server 250 maintains mapping data specifying the EMR systems at which EMRs linked to healthcare providers are stored. The mapping data may be maintained in data store 270. In one embodiment, the mapping data represents a healthcare provider-EMR mapping that specifies the EMRs or data portions thereof mapped to the healthcare provider, and also the locations of the EMRs in multiple EMR systems.

It should be appreciated that such a mapping may be desirable in the scenario that a patient has visits with the same doctor/physician (healthcare provider) consulting at multiple healthcare locations (e.g. hospitals, clinics, etc.) operating with different EMR systems. In such a scenario, the mapping data may indicate the EMR systems operative at different healthcare locations where the doctor/physician (healthcare provider) is consulting.

In step 330, healthcare server 250 receives a request from a healthcare provider to view information related to a patient. The request may be received from one of client systems 210A-210Z by a user/healthcare provider such as a physician/doctor. The request may be received when the physician wants to review the status of his patients or in response to a patient coming for check-up/review with the physician, either in-house or as an out-patient as is well known.

In one embodiment, healthcare server 250 enables the user/physician to specify the details of the current visit of the patient, for example, based on the physical inspection/testing of the patient. In the following description, it is assumed that the user/physician is using client system 210A to send the request specifying the symptoms.

In one embodiment, healthcare server 250 checks for validity of authentication details of the user/physician connecting via client system 210A. If authentication details of the user are found invalid, an authentication failed error message is displayed to the user. In addition, healthcare server 250 may allow the user to retry logging in for a predetermined number of times (which may be pre-configured). Control passes to step 350 if the authentication details of the user are found valid.

In step 350, healthcare server 250 examines the mapping data to identify a set of EMR systems that store a set of EMRs that are linked to the healthcare provider. In one embodiment, healthcare server 250 also retrieves the identified set of EMRs from the respective EMR system by interfacing with integration system 280. The retrieved information related to the patient may include patient information, symptoms of medical problem, details of visits, details of upcoming appointments, doctor's notes, observations, clinical summary, medications, patient documents, care plan, etc.

It may be appreciated that EMR data may also be an aggregation of data from multiple EMR systems. For example, data portions forming the EMR data related to a single patient may be maintained in different EMR systems. EMR data for the single patient is then created by retrieving the various data portions from the different EMR system, according to an aspect of the present disclosure.

In step 370, healthcare server 250 provides access to the set of EMRs using a common user interface. In one embodiment, the common user interface is displayed on a display unit associated with client system 210A. According to an aspect of the present disclosure, the common user interface facilitates a healthcare provider to access data stored in different EMR systems. Control then passes to step 399 where the flowchart ends.

It may be appreciated that the healthcare system 150 may provide the above described features to client systems 210A-210Z in any convenient manner. The description is continued illustrating the manner in which aspects of the present disclosure are implemented in a common graphical user interface in an embodiment.

### 4. Sample User Interfaces

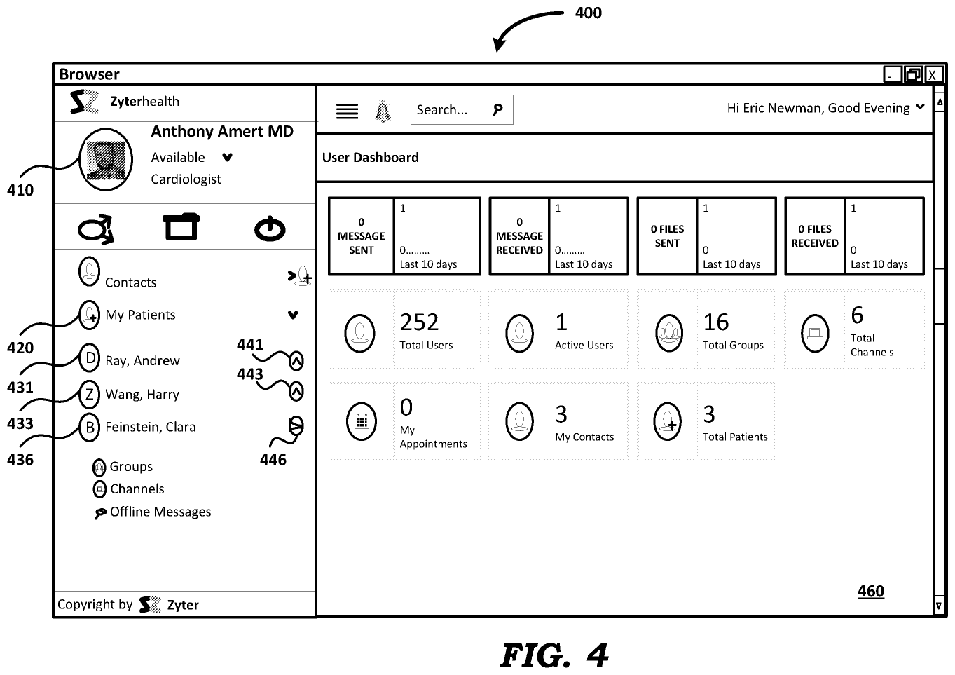

FIGS. 4 and 5 depicts portions of a common graphical user interfaces (GUI) providing data aggregation from multiple EMR systems in an embodiment. A GUI entails aspects such as receiving of inputs from users for the application and displaying the outputs generated by the application, as is well known in the relevant arts. Furthermore, it may be appreciated that GUIs may be customized based on various factors (for example, based on whether the requesting client system is a mobile device or a desktop computer, based on the preferences of the participants etc.).

Display area 400 of FIG. 4 (and also display area 500 of FIG. 5) represents a portion of a user interface displayed on a display unit (not shown) associated with one of client systems (assumed to be 210A, for illustration). In one embodiment, display area 400 corresponds to a web page rendered by a browser executing on the client system. Web pages are provided by healthcare server 250 in response to a user sending appropriate requests (for example, by specifying corresponding URLs in the address bar) using the browser.

The description is continued assuming that display area 400 (common user interface) is being displayed on a system used by the healthcare provider in response to the authentication of the healthcare provider being successful and after the patient details are identified from multiple EMR systems (step 350 of FIG. 3).

Display area 410 indicates the name (“Anthony Amert”) of the physician/healthcare provider who has been successfully authenticated. Display area 420 indicates a list of patients associated with the healthcare provider. The list of patients is shown containing three patients, whose corresponding names are shown in display areas 431, 433, and 436. Each of the patient names may be provided as selectable links/buttons to enable the user/healthcare provider to select a patient of interest.

Each of the patient names is shown associated with an icon (display areas 441, 443, and 446) indicating the source EMR system from which the EMR of the corresponding patient has been retrieved. It may be observed that display area 441 and 443 display the same icon indicating that the details of the patients shown in display area 431 and 433 have been retrieved from the same/first EMR system, while a different icon in display area 446 indicates that the details of the patient shown in display area 436 has been retrieved from a second EMR system different from the first EMR system.

Display area 460 shows the details of a user dashboard containing information relevant to the user/healthcare provider such as his/her contacts, appointments, total patients, total groups, total channels, total messages received/sent, files received/sent, etc.

Thus, a list of patients is presented to the healthcare provider using the common user interface shown in display area 400. As noted above, the healthcare provider may indicate a selection of a patient of interest (e.g. patient under check-up) from the list of patients. In one embodiment, the healthcare provider selects/clicks the selectable link/button provided in each of display area 431, 433 and 436. The description is continued assuming that the healthcare provider has selected the patient (“Feinstein, Clara”) indicated in display area 431.

Referring to FIG. 5, display area 500 there is displayed in response to the healthcare provider selecting the patient name shown in display area 431 of FIG. 4. Accordingly, display area 510 is shown indicating the name (“Feinstein, Clara”) of the selected patient. Display area 520 specifies the details of the selected patient. For convenience, the details of the selected patient are presented under various tabs (display area 530) such as Patient Info, Visit Summary, Observations, Medications, Clinical Summary and Health Tracker.

Display area 535 indicates that the user has already selected the “Visit Summary” tab from the tabs shown in display area 530. Display area 540 accordingly is shown specifying the details of various physicians visited by the selected patient. Thus, the operation of the common user interface shown in FIGS. 4 and 5 facilitates the healthcare provider to access/view the aggregated EMR data related to a patient.

It may be appreciated that the above features of the present disclosure may be provided by healthcare server 250 based on data maintained in data stores 235A-235B/270. Sample data that may be maintained in such data stores is described below with examples.

### 5. Sample Data

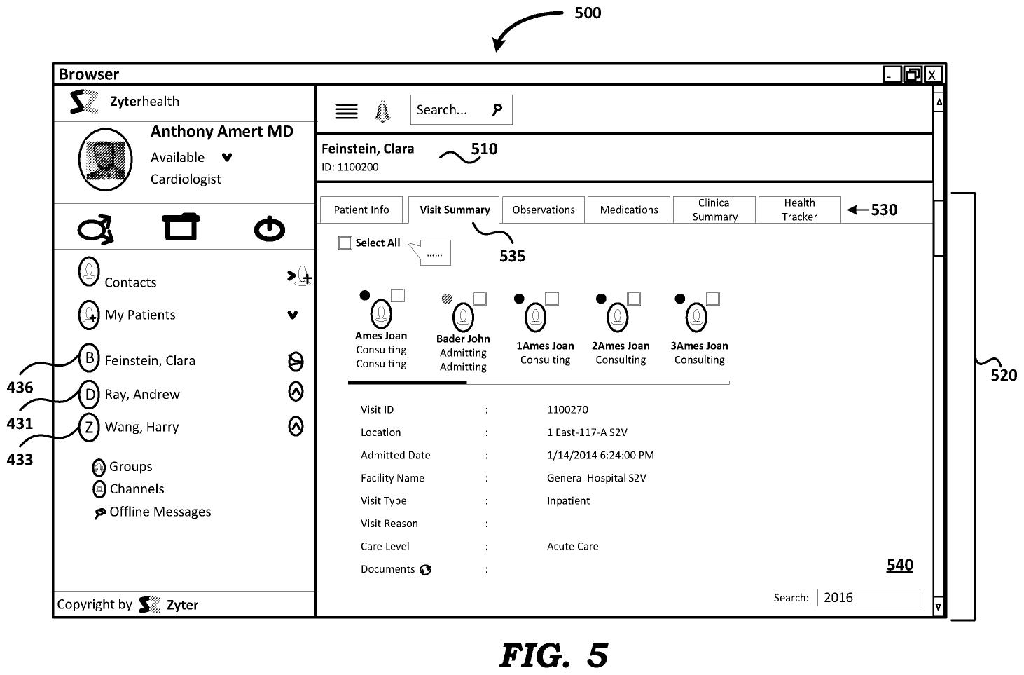

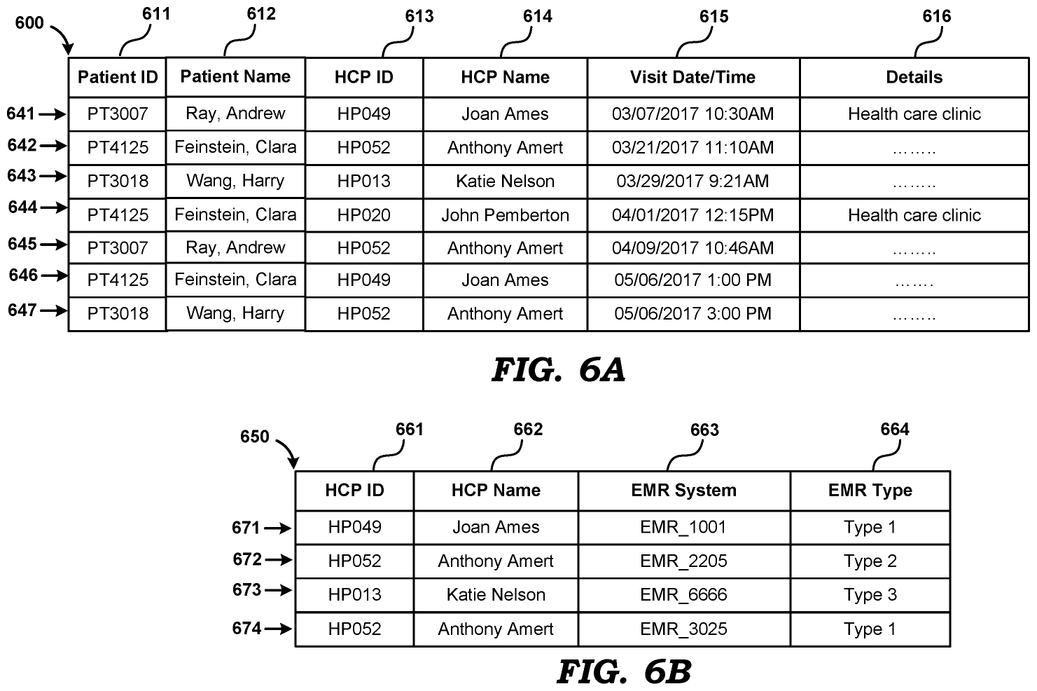

FIG. 6A depicts portions of a visit summary data specifying the details of interactions between patients and healthcare providers in one embodiment. FIG. 6B depicts portions of a mapping data specifying the EMR systems linked to healthcare providers in one embodiment. For illustration, the visit summary data and mapping data are assumed to be maintained in the form of tables in data store 270. However, in alternative embodiments, the visit summary data and/or mapping data may be maintained according to other data formats (such as files according to extensible markup language (XML), etc.) and/or using other data structures (such as lists, trees, etc.), as will be apparent to one skilled in the relevant arts by reading the disclosure herein.

Referring to FIG. 6A, table 600 depicts visit summary data specifying the details of visits of different patients to the healthcare organization. In particular, column 611 “Patient ID” specifies a unique identifier associated with a patient and column 612 “Patient Name” specifies the name of the patient. Columns 613 “HCP ID” and 614 “HCP Name” respectively specify a unique identifier and the name of the healthcare provider who attended to (and provided a corresponding healthcare service) to the patient. Column 615 “Visit Date/Time” specifies the date and time of the visit of the patient and column 616 “details” specifies the details of the visit. Only a sample number of columns is shown in table 600, though in alternative embodiments, a large number of additional columns may be present to indicate additional details of the visit of the patient.

Each of rows 641-647 specifies the details of a corresponding visit by patients. It may be readily observed that rows 642, 645 and 647 specify the details of the visits by three different patients to the same healthcare provider having identifier “HP052” (name “Anthony Amert”). Similarly, other rows specify the details of other interactions between patients and other healthcare providers.

Referring to FIG. 6B, table 650 portions of a mapping data specifying the EMR systems linked to healthcare providers. In particular, columns 661 “HCP ID” and 662 “HCP Name” respectively specify a unique identifier and the name of the healthcare provider, while columns 663 “EMR System” and 664 “EMR Type” respectively specify the identity and type of an EMR system linked to the healthcare provider.

Thus, each of rows 671-674 specifies a corresponding mapping between a healthcare provider and an EMR system that stores EMRs of patients related to the healthcare provider. It may be readily observed that rows 672 and 674 indicate the different EMR systems that are linked to healthcare provider having identifier “HP052” (name “Anthony Amert”). Similarly, other rows specify the details of other EMR systems at which EMRs linked to healthcare providers are stored.

In response to receiving a request from a healthcare provider (assumed to be “HP052” named “Anthony Amert” for illustration) using the interfaces of FIGS. 4 and 5, healthcare server 250 first examines the data of table 650 to determine the various EMR systems linked to the requesting healthcare provider. Healthcare server 250 also inspects the data of table 600 and determines a list of patients (rows 642, 645, and 647) having interactions with the requesting healthcare provider and sends the determined list to be displayed as part of the common user interface.

Upon the healthcare provider selecting a patient of interest (assumed to be “PT4125” named “Feinstein, Clara” for illustration), healthcare server 250 interfaces with the EMR systems indicated in rows 672 and 674 of table 650 and retrieves the EMRs (or portions thereof) containing information related to the patient of interest. Healthcare server 250 then provides access to the retrieved EMRs/portions using the common user interface as described above with respect to FIGS. 4 and 5.

Healthcare server 250 also identifies healthcare providers having responsibility for the patient, that is, the healthcare providers who have treated the patient during earlier visits. As such, healthcare server 250 identifies the healthcare providers “HP052”, “HP020” and “HP049” in rows 642, 644 and 646 as having responsibility for the patient “PT4125”. According to an aspect of the present disclosure, healthcare server 250 facilitates the requesting healthcare provider (“HP052” named “Anthony Amert”) to collaborate with one or more of the other identified healthcare providers.

Thus, healthcare system 150 provides effective data aggregation of different EMR systems using the visit summary data of table 600 and the mapping data of table 650. The manner in which healthcare system 150 may be implemented is described below with examples.

### 6. Example Implementation

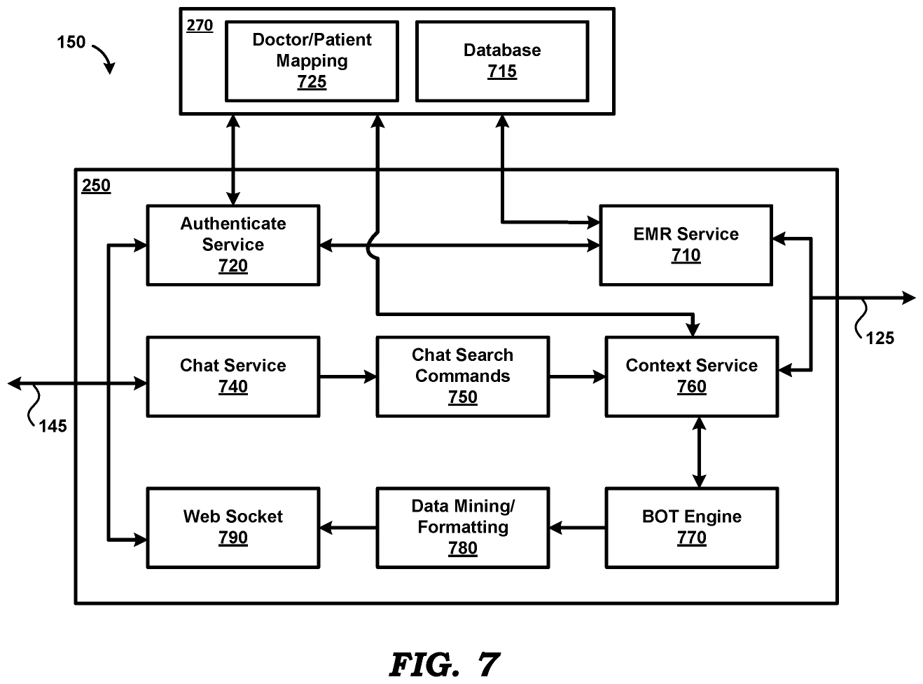

FIG. 7 is a block diagram illustrating an example implementation of a healthcare system (150). The example implementation is shown in the form of a functional architecture containing multiple functional blocks implemented in various systems (such as healthcare server 250 and data store 270) belonging to healthcare system (150).

Healthcare server 250 is shown containing EMR service 710, authenticate service 720, chat service 740, chat search commands 750, context service 760, BOT engine 770, data mining/formatting 780 and web socket 790, while data store 270 is shown containing database 715 and doctor/patient mapping 725. It may be appreciated that in alternative embodiments, the various blocks/components may be implemented on multiple servers/data stores (which then together form healthcare system 150) as will be apparent to one skilled in the relevant arts. Each of the blocks of the healthcare system is described in detail below.

EMR service 710 facilitates accessing of data from external systems such as 290A-290B. For example, EMR service 710 may interact with the external systems such as integration system 280 to request for authentication/authorization tokens for accessing the external EMR/EHR data. Such authentication tokens may be stored in database 715 and later used when accessing the external EMR/EHR data.

Database 715 facilitates storing/retrieving of data by other blocks in the healthcare system based on SQL queries. Some examples of data that may be maintained in database 715 include, but not limited to, authentication token from EMR service 710, authentication data of the users/healthcare providers from authenticate service 720, doctor/patient mapping data 725, chat history of the participants from chat service 740 and context data from context service 760.

Authenticate service 720 facilitates authentication and/or authorization of the users/healthcare providers to avail the services of the healthcare system of the present disclosure. Authenticate service 720 may employ standard authentication and/or authorization techniques well known in the relevant art such as Active Directory, Single Sign On, Database and LDAP. Furthermore, security technologies known in the relevant art (such as SSL) may be used by authenticate service 720 during authentication and/or authorization. Authenticate service 720 may maintain user related data in database 715 for providing the authentication and/or authorization service.

Doctor/patient mapping 725 represents a mapping (data) between patients and doctors such as list of doctors consulted by a patient and/or list of patients treated by a doctor. An example of such a mapping is the visit summary data shown in table 600 of FIG. 6A. The doctor/patient mapping 725 may be updated periodically (for example, based on periodical examination of external EMR/EHR data). In addition, the mapping data may be updated by physician/healthcare providers during the visits by the patients.

Upon successful authentication and/or authorization of the user/healthcare provider, authenticate service 720 determines a list of patients handled by the authenticated user based on the information in doctor/patient mapping data 725 (as described in detail above). Authenticate service 720 then sends for display (for example, by sending the details to requesting client system 210A) the corresponding list of patients, thereby allowing the healthcare provider to access the profiles of his/her patients (including complete medical history, information related to last visit, appointments etc.).

Chat service 740 enables chat/collaboration among multiple participants of the healthcare system. Choosing/selecting a chat option for a particular patient enables an authenticated participant to discuss about a patient with other participants. In an example implementation, upon choosing the chat option, chat service 740 initiates a chat session by creating a group with all the participants (healthcare providers) having responsibility for the patient. Chat service 740 may use doctor-patient mapping data 725 for initiating the chat session. In addition, chat service 740 may store a chat history specifying the details of chats performed earlier in database 715. Chat service 740 also may identify and forward search commands issued by the participants during a chat session to chat search commands 750.

Chat search commands 750 enables the participants in a chat session to issue search commands for required information such as patient information, last vital information, patient/doctor visits, files etc. in chat history (stored in database 715) and/or external EMR/EHR databases and to and receive/view the results of the search commands. Chat search commands 750 may accordingly interact with context service 760 to perform the desired searches indicated by the issued search commands, receive the results of the searches and provide the results as corresponding responses to the issued search commands.

Context service 760 enables other blocks/components of healthcare system to retrieve required data from database 715 and and/or external EMR/EHR databases based on a context. The context may be related to a specific patient, a specific healthcare provider, and/or the specific background (e.g. triaging, chat, etc.) in which the context service has been requested. Context service 760 receives requests from chat search commands 750 or BOT engine 770 indicating the specific data to be retrieved. Context service 760 may then retrieve the specific data requested from database 715 and and/or external EMR/EHR databases (235A-235B) and forwards the retrieved data as corresponding responses to the requests.

BOT engine 770 provides an execution environment for executing bots/automated programs that perform a pre-defined set of actions, possibly by interacting with other systems such as client systems 210A-210Z. Some bots run automatically, while others only execute commands when they receive specific inputs. Examples of bots are web crawlers, chat room bots etc. Some of the bots may also facilitate predictive analytics using predictive algorithms. For example, in the field of healthcare, a patient info bot may pull the complete information/data of a patient from a database (which can be EMR/EHR and may act on the data to find some relevant/meaningful information and advice a doctor with suggestions about the areas of treatment).

In one embodiment, a chat bot (executing in BOT engine 770) performs predictive analytics (using predictive algorithms) on chat/collaboration history, chat context and commands given by participants during a chat/collaboration session, and render the results/suggestions to the participants. For example, when a participant sends a message (typically in the form of text) in a chat session indicating that a patient is suffering from high fever, headache, rash, muscle and joint pain, a chat bot may search for the symptoms in chat/collaboration history (related to all patients) and may thereafter arrive at the suggestion that the patient might be suffering from dengue fever (diagnosis). The chat bot may also suggest possible medications for the diagnosis.

Data mining/formatting 780 provides both mining and formatting services such as formatting/modifying the results of the predictive analytics of bot engine 770 according to the requirements of client systems 210A-210Z, and send the formatted output (e.g. in the form of a report) to the client systems via web socket 790, thereby enabling client systems 210A-210Z to render the formatted output to the participants.

In addition, data mining/formatting 780 may also inspect the data received from bot engine 770 and determine additional insights/suggestions using mining algorithms well known in the art. For example, data mining/formatting 780 may identify additional data to be included to the data received from bot engine 770.

Web socket 790, well-known in the relevant arts, is a computer communications protocol, providing full-duplex communication channels over a single TCP connection, and facilitates peer to peer and client-server communication. Web socket 790 facilitates the collaborative communication among the participants (here healthcare providers). Also, web socket 790 facilitates data mining/formatting 780 to send the results/suggestions received from bot engine 770 to participants using client systems 210A-210Z.

It may be appreciated that the various components of FIG. 7 facilitate a healthcare system (150) to provide several aspects of the present disclosure. According to an aspect of the present disclosure, EMR service 710 facilitates the EMR data related to a patient to be aggregated from multiple/external EMR/EHR databases via integration system 280. As noted above, EMR service 710 may interact with integration system 280 to request for authentication/authorization tokens for accessing the external EMR data.

EMR service 710 also facilitates users/healthcare providers to access/view the aggregated EMR data related to a patient using a common user interface. EMR service 710 also facilitates with drill-down capabilities wherein the user is enabled to view the aggregated EMR data at various levels.

According to another aspect of the present disclosure, the doctor-patient mapping data and a doctor-EMR mapping data maintained in database 715 facilitates the retrieval of aggregated EMR data related to a patient. The doctor-EMR mapping data specifies for each healthcare provider, the associated EMRs of patients or data portions thereof and the location of the EMRs/data portions in multiple EMR systems. EMR service 710 is accordingly facilitated to retrieve (via integration system 280) the indicated data portions from the EMR systems and to form the aggregated EMR data for the patient.

It may be appreciated that the operation of FIGS. 2 through 7 facilitates the healthcare system to provide effective data aggregation from multiple different EMR systems.

It may be further appreciated that the features of the present disclosure are described above assuming that the healthcare system is implemented on a single server (healthcare server 250 of FIG. 2). However, healthcare systems are typically implemented using multiple servers and data stores. The manner in which a healthcare system may be provided using a cloud infrastructure is described below with examples.

### 7. Healthcare System Using Cloud Infrastructure

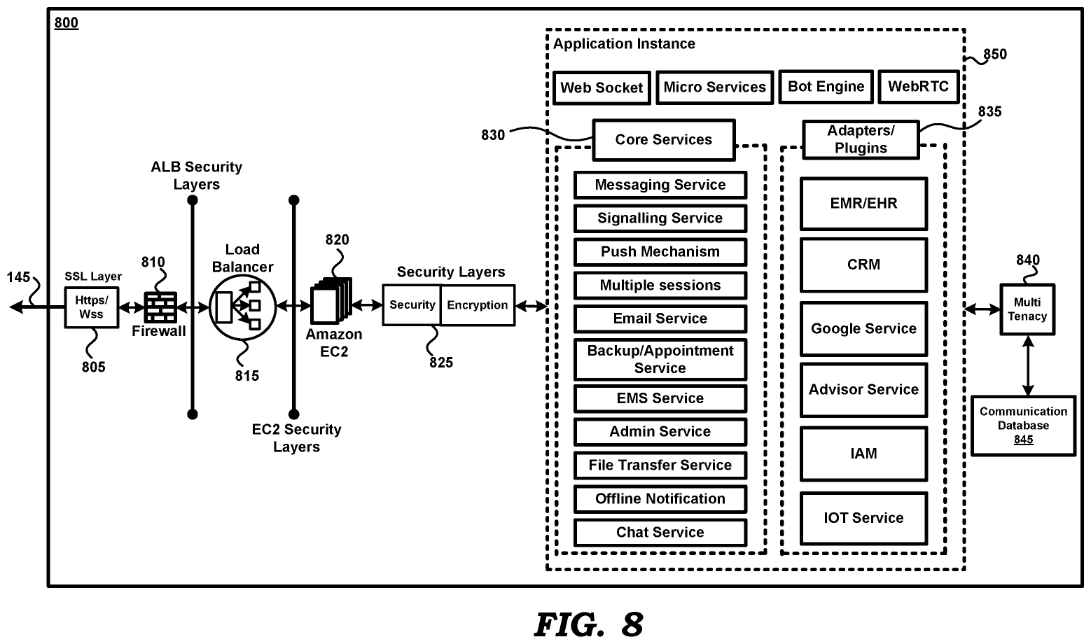

FIG. 8 is a block diagram illustrating the manner in which a healthcare system is provided using a cloud infrastructure in an embodiment. Specifically, the healthcare system is shown implemented using AWS (Amazon Web Services) framework 800 available from Amazon™. Accordingly, the block diagram is shown containing SSL (Secure Sockets layer) 805, firewall 810, load balancer 815, Amazon Elastic Compute Cloud (Amazon EC2) 820, security layers 825, application server (instance) 850, and multi tenancy 840.

It should be appreciated that the healthcare system is implemented using multiple (typically, 50-100) application servers, though only a single application server 850 is shown in FIG. 8 for clarity.

SSL 805 is a standard security technology for establishing an encrypted link between application server 850 and any of client systems 210A-210Z. Firewall 810 is a network security device that monitors traffic to or from a network. Firewall 810 also allows or blocks traffic based on a defined set of security rules. Amazon EC2 820 is a web service that provides secure, resizable compute capacity on the cloud. Application Load Balancer (ALB) 815 distributes the user requests among the various application servers for reasons such as scalability, fault tolerance, etc. Security layers 825 provide additional level of security for the healthcare system in addition to SSL 805 and firewall 810. Multi tenancy 840 handles the tenancy of healthcare system in Amazon EC2 820.

Application server 850 is shown containing core services 830 and adapters/plugins 835 (in addition to associated components such as web socket, micro services and bot engine described above with respect to FIG. 6). Core services 830 facilitate the interaction among one or more application servers and also the interaction with client systems 210A-210Z, while adapters/plugins 835 facilitate application server to interact with external systems (via integration system 280). Some of core services 830 and adapters/plugins 835 are described in detail below.

Messaging service enables messages to be sent among application servers and/or client systems 210A-210Z. Such messages may be encrypted using user keys. Signaling service is used to provide audio/video calls for collaboration among participants on client systems 210A-210Z. Such audio/video calls may be provided using WebRTC, WebSocket and Amazon EC2 820. Push mechanism pushes messages such as alerts or emergency notifications to the client system used by the appropriate user (here doctor/nursing staff). Multiple sessions service enables a user to login into multiple client systems simultaneously. Email service enables a user to compose and send emails including any attachments if any.

Backup/appointment service enables user to assign another user as back up (in cases of emergency) on his/her unavailability. In addition, backup/appointment service sends alerts or emergency notifications to another user assigned as back up. Furthermore, backup/appointment service enables recording of appointments for patient's consultations. As an example, recording of appointments includes modifying date, time and doctor mapped for consultation.

EMS (Enterprise Messaging Service) service provides multi factor authentications of users. For example, a two factor authentication (two levels of verification) of login details of a user may be provided. Details of such two levels of verification are sent via emails, SMS (Short Messaging Service), etc. Admin service enables a user to fetch data from PHI (Protected Health Information) systems via integration system 280. File transfer service enables a user to send/share documents to another user in real time, for example, during collaboration. In addition, file transfer service may encrypt the documents before sending/sharing.

Offline notification service sends alerts to a user notifying emergency when client systems 210A-210Z are in offline mode. Offline notification service uses Firebase or Apple Push Notification Service (APNs) to send the alerts, for example, in the form of alarm beeps. Chat service enables chat based collaboration among multiple participants including sending of private messages, creating chat rooms/groups and storing the chat history.

EMR/EHR adapter enables retrieving of EMR of patients from external EMR/EHR systems via integration system 280. Exemplary data in EMR includes Patient documents, medications, Patient info, care plan, doctor notes, observations, clinical summary and visits details. CRM adapter facilitates data to be retrieved from an external CRM system via integration system 280. Google service adapter facilitates application server 850 to interface with Google™ services. Advisor service adapter facilitates interactions with third party bots to retrieve suggestions and recommendations. IAM (Identity and Access Management) adapter facilitates interactions with external servers to perform authentication and/or authorization of the users/healthcare providers in the healthcare system.

IoT service adapter facilitates interactions with IoT devices such as heart monitors. Such interaction may facilitate application server 850 to determine whether heartbeat of a patient monitored through an IoT device records a lower value or a higher value than the standard thresholds, and then send an alert to the appropriate medical staff (nurse/doctor).

Additional components such as Micro services, WebRTC, Web sockets and Bot engine may be associated with application server 850. Micro services are back end services or self-sustained services and these can be any plug and play services. For example, in healthcare industry, the micro services render the patient data required by the doctors. WebRTC ((Web Real-Time Communication) provides web browsers and mobile applications with real-time communication (RTC) via simple application programming interfaces (APIs). WebRTC allows audio and video communication to work inside web pages by allowing direct peer-to-peer communication, eliminating the need to install plugins or download native apps. Web socket and Bot engine are described above with respect to FIG. 6.

Communication database 845 represents a non-volatile (persistent) storage facilitating storage and retrieval of data by applications executing in other systems such as application server 850. Communication database 845 may be implemented as a database server using relational database technologies and accordingly provide storage and retrieval of data using structured queries such as SQL (Structured Query Language). Alternatively, or in addition, communication database 845 may be implemented as a file server providing storage and retrieval of data in the form of files organized as one or more directories, as is well known in the relevant arts.

It should be appreciated that the features described above can be implemented in various embodiments as a desired combination of one or more of hardware, executable modules, and firmware. The description is continued with respect to an embodiment in which various features are operative when executable modules are executed.

### 8. Digital Processing System

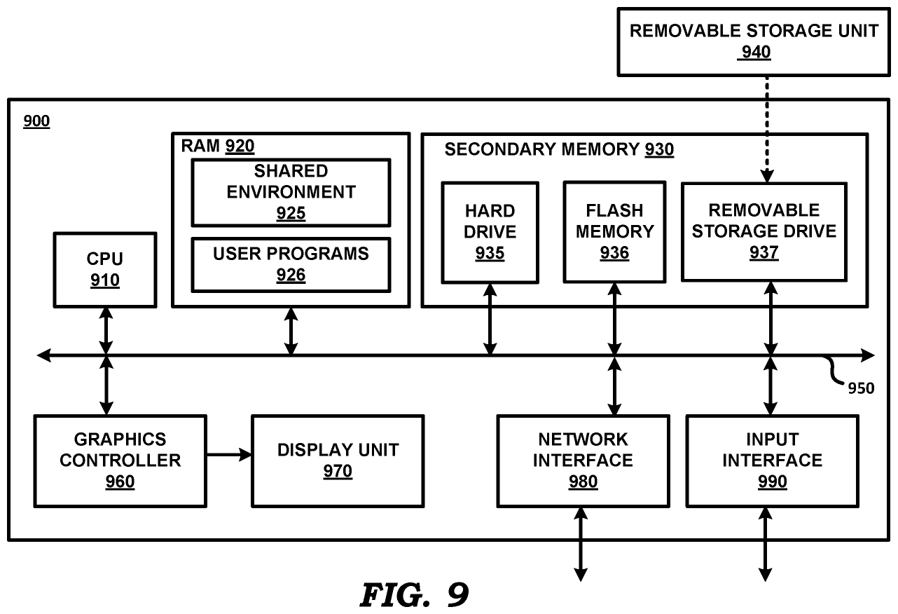

FIG. 9 is a block diagram illustrating the details of digital processing system 900 in which various aspects of the present disclosure are operative by execution of appropriate executable modules. Digital processing system 900 corresponds to healthcare server 250 or application server 850.

Digital processing system 900 may contain one or more processors such as a central processing unit (CPU) 910, random access memory (RAM) 920, secondary memory 930, graphics controller 960, display unit 970, network interface 980, and input interface 990. All the components except display unit 970 may communicate with each other over communication path 950, which may contain several buses as is well known in the relevant arts. The components of FIG. 9 are described below in further detail.

CPU 910 may execute instructions stored in RAM 920 to provide several features of the present disclosure. CPU 910 may contain multiple processing units, with each processing unit potentially being designed for a specific task. Alternatively, CPU 910 may contain only a single general-purpose processing unit.

RAM 920 may receive instructions from secondary memory 930 using communication path 950. RAM 920 is shown currently containing software instructions constituting shared environment 925 and user programs 926. Shared environment 925 includes operating systems, device drivers, virtual machines, etc., which provide a (common) run time environment for execution of user programs 926.

Graphics controller 960 generates display signals (e.g., in RGB format) to display unit 970 based on data/instructions received from CPU 910. Display unit 970 contains a display screen to display the images defined by the display signals (e.g. portions of the GUI shown in FIGS. 4 and 5). Input interface 990 may correspond to a keyboard and a pointing device (e.g., touch-pad, mouse) that may be used to provide appropriate inputs (e.g. inputs specified in the user interfaces of FIGS. 4 and 5). Network interface 980 provides connectivity to a network (e.g., using Internet Protocol), and may be used to communicate with other systems (of FIG. 1) connected to the network.

Secondary memory 930 may contain hard drive 935, flash memory 936, and removable storage drive 937. Secondary memory 930 may store the data (for example, portions of visit summary data of FIG. 6A and mapping data of FIG. 6B) and software instructions (for implementing the flowchart of FIG. 3 and to provide several features of the present disclosure), which enable digital processing system 900 to provide several features in accordance with the present disclosure. The code/instructions stored in secondary memory 930 either may be copied to RAM 920 prior to execution by CPU 910 for higher execution speeds, or may be directly executed by CPU 910.

Some or all of the data and instructions may be provided on removable storage unit 940, and the data and instructions may be read and provided by removable storage drive 937 to CPU 910. Removable storage unit 940 may be implemented using medium and storage format compatible with removable storage drive 937 such that removable storage drive 937 can read the data and instructions. Thus, removable storage unit 940 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 940 or hard disk installed in hard drive 935. These computer program products are means for providing software to digital processing system 900. CPU 910 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as secondary memory 930. Volatile media includes dynamic memory, such as RAM 920. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 950. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

### 9. Conclusion

While various embodiments of the present disclosure have been described above, it should be understood that they have been presented by way of example only, and not limitation. Thus, the breadth and scope of the present disclosure should not be limited by any of the above-described exemplary embodiments, but should be defined only in accordance with the following claims and their equivalents.

It should be understood that the figures and/or screen shots illustrated in the attachments highlighting the functionality and advantages of the present disclosure are presented for example purposes only. The present disclosure is sufficiently flexible and configurable, such that it may be utilized in ways other than that shown in the accompanying figures.

Further, the purpose of the following Abstract is to enable the Patent Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present disclosure in any way.

What is claimed is:

1. A method comprising:
maintaining, via a healthcare system, mapping data specifying the Electronic Medical Record (EMR) systems at which EMRs linked to healthcare providers are stored, wherein each EMR contains information related to a corresponding patient;
receiving, via the healthcare system, a request from a healthcare provider to view information related to a patient;
examining, via the healthcare system, the mapping data to identify a set of EMR systems that store a set of EMRs that are linked to the healthcare provider and contain information related to the patient; and
providing, via the healthcare system, access to the set of EMRs using a common user interface;
wherein the method is configured to facilitate data aggregation from EMR systems via the healthcare system.

2. The method of claim 1, further comprising:
maintaining, via the healthcare system, visit summary data specifying the details of interactions between patients and healthcare providers;
identifying, via the healthcare system based on the visit summary data, a set of patients having interactions with the healthcare provider; and
providing, via the healthcare system, the set of patients, wherein the request is received in response to the healthcare provider selecting the patient from the set of patients.

3. The method of claim 2, wherein the information for the patient is stored in a first EMR system and the information for a second patient is stored in a second EMR system, wherein the providing further comprises:
displaying, via the healthcare system, a first icon representing the first EMR system in association with the patient, and a second icon representing the second EMR system in association with the second patient.

4. The method of claim 1, wherein a first portion and a second portion of the information for the patient is stored respectively in a first EMR system and a second EMR system, wherein the providing further comprises:
displaying, via the healthcare system, both of the first portion and the second portion together as part of the common user interface.

5. The method of claim 4, wherein the first portion indicates that a first set of healthcare providers have responsibility for the patient and the second portion indicates that a second set of healthcare providers have responsibility for the patient,
the method further comprising facilitating, via the healthcare system, the healthcare provider to collaborate with the first set of healthcare providers and the second set of healthcare providers.

6. A method comprising:
sending, via a client system, a request to view information related to a patient; and
providing, via the client system using a common user interface, access to a set of Electronic Medical Records (EMRs) containing information related to the patient, the set of EMRs being stored on different EMR systems;
wherein the method is configured to facilitate a healthcare provider to access data stored in different EMR systems via the client system.

7. The method of claim 6, further comprising providing, via the client system, a set of patients having interactions with the healthcare provider, wherein the request is received in response to the healthcare provider selecting the patient from the set of patients.

8. The method of claim 7, wherein the information for the patient is stored in a first EMR system and the information for a second patient is stored in a second EMR system, wherein the providing further comprises:
displaying, via the client system, a first icon representing the first EMR system in association with the patient, and a second icon representing the second EMR system in association with the second patient.

9. The method of claim 6, wherein a first portion and a second portion of the information for the patient is stored respectively in a first EMR system and a second EMR system, wherein the providing further comprises:
displaying, via the client system, both of the first portion and the second portion together as part of the common user interface.

10. The method of claim 9, wherein the first portion indicates that a first set of healthcare providers have responsibility for the patient and the second portion indicates that a second set of healthcare providers have responsibility for the patient,
the method further comprising facilitating, via the client system, the healthcare provider to collaborate with the first set of healthcare providers and the second set of healthcare providers.

11. A tangible non-transitory machine readable medium comprising instructions executable by one or more processors for implementing one or more operations in a healthcare system for facilitating data aggregation from different EMR systems, the operations comprising:
maintaining, via a healthcare system, mapping data specifying the Electronic Medical Record (EMR) systems at which EMRs linked to healthcare providers are stored, wherein each EMR contains information related to a corresponding patient;
receiving, via the healthcare system, a request from a healthcare provider to view information related to a patient;
examining, via the healthcare system, the mapping data to identify a set of EMR systems that store a set of EMRs that are linked to the healthcare provider and contain information related to the patient; and
providing, via the healthcare system, access to the set of EMRs using a common user interface;

12. The non-transitory machine readable medium of claim 11, the operations further comprising:
maintaining, via the healthcare system, visit summary data specifying the details of interactions between patients and healthcare providers;
identifying, via the healthcare system based on the visit summary data, a set of patients having interactions with the healthcare provider; and
providing, via the healthcare system, the set of patients, wherein the request is received in response to the healthcare provider selecting the patient from the set of patients.

13. The non-transitory machine readable medium of claim 12, wherein the information for the patient is stored in a first EMR system and the information for a second patient is stored in a second EMR system, wherein the providing further comprises:
displaying, via the healthcare system, a first icon representing the first EMR system in association with the patient, and a second icon representing the second EMR system in association with the second patient.

14. The non-transitory machine readable medium of claim 11, wherein a first portion and a second portion of the information for the patient is stored respectively in a first EMR system and a second EMR system, wherein the providing further comprises:
displaying, via the healthcare system, both of the first portion and the second portion together as part of the common user interface.

15. The non-transitory machine readable medium of claim 14, wherein the first portion indicates that a first set of healthcare providers have responsibility for the patient and the second portion indicates that a second set of healthcare providers have responsibility for the patient,
the operations further comprising facilitating, via the healthcare system, the healthcare provider to collaborate with the first set of healthcare providers and the second set of healthcare providers.

16. A digital processing system comprising:
a memory to store instructions;
one or more processors to execute the instructions stored in the memory to cause the digital processing system to perform the actions of:
maintaining, via the digital processing system, mapping data specifying the Electronic Medical Record (EMR) systems at which EMRs linked to healthcare providers are stored, wherein each EMR contains information related to a corresponding patient;
receiving, via the digital processing system, a request from a healthcare provider to view information related to a patient;
examining, via the digital processing system, the mapping data to identify a set of EMR systems that store a set of EMRs that are linked to the healthcare provider and contain information related to the patient; and
providing, via the digital processing system, access to the set of EMRs using a common user interface;
wherein the digital processing system is configured to facilitate data aggregation from different EMR systems.

17. The digital processing system of claim 16, further performing the actions of:
maintaining, via the digital processing system, visit summary data specifying the details of interactions between patients and healthcare providers;
identifying, via the digital processing system based on the visit summary data, a set of patients having interactions with the healthcare provider; and
providing, via the digital processing system, the set of patients, wherein the request is received in response to the healthcare provider selecting the patient from the set of patients.

18. The digital processing system of claim 17, wherein the information for the patient is stored in a first EMR system and the information for a second patient is stored in a second EMR system, wherein for the providing, the digital processing system performs the actions of:
displaying, via the digital processing system, a first icon representing the first EMR system in association with the patient, and a second icon representing the second EMR system in association with the second patient.

19. The digital processing system of claim 16, wherein a first portion and a second portion of the information for the patient is stored respectively in a first EMR system and a second EMR system, wherein for the providing, the digital processing system performs the actions of:
displaying, via the digital processing system, both of the first portion and the second portion together as part of the common user interface.

20. The digital processing system of claim 14, wherein the first portion indicates that a first set of healthcare providers have responsibility for the patient and the second portion indicates that a second set of healthcare providers have responsibility for the patient,
the digital processing system further performing the actions of facilitating, via the digital processing system, the healthcare provider to collaborate with the first set of healthcare providers and the second set of healthcare providers.
